(19) United States
(12) Patent Application Publication
(10) Pub. No.: US 2004/0254518 A1
(43) Pub. Date: Dec. 16, 2004

(54) Device to promote blood flow into the myocardium

(76) Inventor: Sun Lee, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/811,424

(22) Filed: Mar. 26, 2004

Related U.S. Application Data
(60) Provisional application No. 60/458,496, filed on Mar. 27, 2003.

(51) Int. Cl. A61M 5/00; A61F 2/06

(52) U.S. Cl. 604/8; 623/1.42

(57) ABSTRACT

The present invention relates to medical devices that are designed to promote blood flow from the left ventricle into the myocardium in order to revascularize an ischemic myocardium in the event of left coronary artery occlusion. Such devices are characterized by a design that facilitates blood flow while minimizing clot formation during and after placement. In addition, the device can be easily implanted with minimally invasive surgical techniques.

### CROSS-REFERENCE TO RELATED APPLICATION

This application claims the benefit of priority under 35 U.S.C. § 119(e) of U.S. Provisional Patent Application 60/458,496 filed Mar. 27, 2003, which is hereby incorporated by reference.

### TECHNICAL FIELD

The present invention relates to medical devices that are designed to promote blood flow from the left ventricle into the myocardium in order to revascularize an ischemic myocardium in the event of left coronary artery occlusion.

### BACKGROUND ART

Under normal conditions, the mammalian heart stores oxygenated blood in the left ventricular chamber at all times. However, in the event of a coronary arterial occlusion, the myocardial muscles become deprived of oxygen-rich ventricular blood due to a rigid endocardium. Remedies to treat such disorders are numerous and include: simple re-vascularization of the ischemic myocardium by a internal mammary artery implantation into the myocardium (Vineberg, A. M., Can. Med. Assoc.(1958) 78:871-979), end-side internal mammary circumflex anastomosis or circumflex arterial replacement (Thai, A. P., et al., Surgery (1956) 40:1023-1029; Absolon, K. B., et al., Surg Gynec Obst. (1956) 103:183-185; and Lee, S., et al., J Res Inst Med Sci (Korea) (1964) 2:297-299), coronary bypass by end-side anastomosis between an arterial segment or a venous segment to the coronary artery (Favaloro, R. C. G., Ann Thorac Surg (1968) 5:334-339, or the use of laser to generate microscopic pores in the ischemic myocardium (Lee, G., et al., Cardial Clinic. (1985) 3:93-100.

One such remedy is described by Thai, supra, who successfully anastomosed a segment of the circumflex artery to the internal mammary artery without the benefit of a cardiac resuscitation apparatus. Thereafter, Vineberg, supra, drew the attention of the thoracic-surgical world with his ability to successfully implant an internal mammary artery into a myocardial tunnel. Lee, supra, developed a procedure that allows replacement of the circumflex arterial segment in a dog model using combined hypothermia and resuscitation techniques. In particular, dogs were hypothermized, placed in cardiac standstill condition, and a segment of the circumflex artery was replaced with a segment of an internal mammary artery. When the animal was warmed, cardiac resuscitation was applied in order to restore cardiac rhythm. Since this time, the coronary bypass surgery utilized by Favaloro, supra, has become a popular choice for cardiac ischemic cases, and many patients that have received this procedure are now leading a normal life.

More recently, some attention has also been give to the ability to direct ventricular blood to the coronary arterial system by way of a ventriculo-coronary conduit that distributes ventricular blood into the ischemic myocardium (Tweden, K. S., et al., Heart Surg. Forum (2000) 3:47-54; and Suehiro, K., et al., Thorac Cardiovasc Surg (2001) 2:307-315). This technique is based in part on the fact that myocardial circulation in reptiles consists of central channels radiating from the myocardial cavity into the myocardium. The channels supply oxygenated blood to the myocardium directly from the ventricular cavity. In an attempt to recreate this vascular pattern, the authors performed transmural acupuncture to perfuse the myocardium through artificially created channels. However, the blood flow through such channels is not easily controlled.

The present invention relates to devices that attempt to minimize the invasive procedures normally associated with the current treatments to alleviate myocardium ischemia. In a control study, a Straight-Porous (SP) Needle is applied to transmit ventricular blood into the myocardium by penetrating the entire thickness of the myocardial wall 4 to 5 times. However, in most of the SP Needle cases, myocardial revascularization did not occur due to blood clot formations in the myocardium that developed post needling.

Accordingly, there is a need for procedures that can be performed in emergency situations to provide immediate myocardial revascularization when more invasive surgical maneuvers would not be possible. The devices and methods described herein meet this need.

### DISCLOSURE OF THE INVENTION

The present invention provides for an implantable device adapted for external insertion into a heart thereby effecting perfusion of oxygenated blood from the ventricle into the myocardium. The device comprises a shaft having an aperture therethrough, and further comprises a proximal end terminating in a point and a distal end adapted to be detachably secured to the outside of the heart. The shaft further comprises at least one opening into the aperture between the proximal end and the distal end, with the proviso that the aperture is closed at the distal end of the device.

A preferred embodiment of the disclosed invention provides for devices, such as the Horse-Shoe (HS) Needle exemplified herein. When the 5.5 mm long HS Needle is inserted at a right angle into the heart's surface, it is able to successfully transmit ventricular blood into the myocardium without the formation of clots.

The present invention also provides for methods of using the device to restore blood flow to ischemic myocardial tissue. Other aspects of the invention are described throughout the specification.

### BRIEF DESCRIPTION OF THE DRAWINGS

FIG. 1(a) depicts ligation of the left coronary artery as described in the Examples.

FIG. 1(b) depicts the use of the SP needle as described in the Examples.

FIG. 2(a) depicts a representative embodiment of the HS needle.

FIG. 2(b) depicts the placement of the HS needle in the ischemic myocardium as described in the Examples.

FIG. 2(c) depicts an enlarged view of the HS needle after placement.

FIG. 3 is a graphical analysis of the Doppler results as described in the Examples.

FIG. 4 depicts the direction of blood flow through the heart.

FIG. 5 depicts an expanded view of the rectangular area shown in FIG. 4 which encompasses a portion of the left ventricle (V) and the myocardium (M) showing alternative embodiments of the device after insertion; FIG. 5(a) depicts the HS needle described in the Examples, whereas FIGS. 5(b) and 5(c) depict alternative shapes. FIGS. 5(d) and 5(e) show alternate embodiments of the devices depicted in FIGS. 5(b) and 5(c), respectively, which have multiple openings along the central shaft.

FIG. 6 depicts an alternate embodiment of the HS device. As shown, additional side holes a and b may also be present. Also as shown, the bevel of the needle is only half exposed to the ventricular blood stream. Representative width (1.8-2 mm) and length (6 mm) dimensions for the rat model are also depicted.

### MODES OF CARRYING OUT THE INVENTION

The invention described herein relates to medical devices designed to promote blood flow from the left ventricle into the myocardium. The disclosed device can be used to revascularize an ischemic myocardium in the event of left coronary artery occlusion.

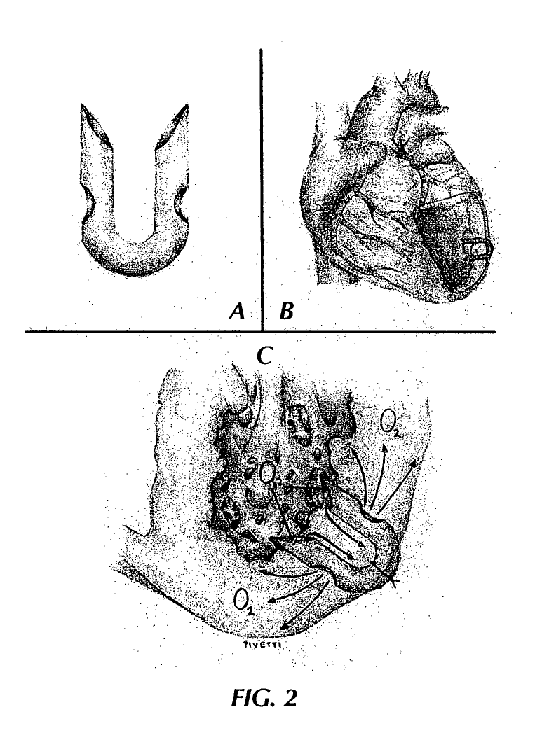

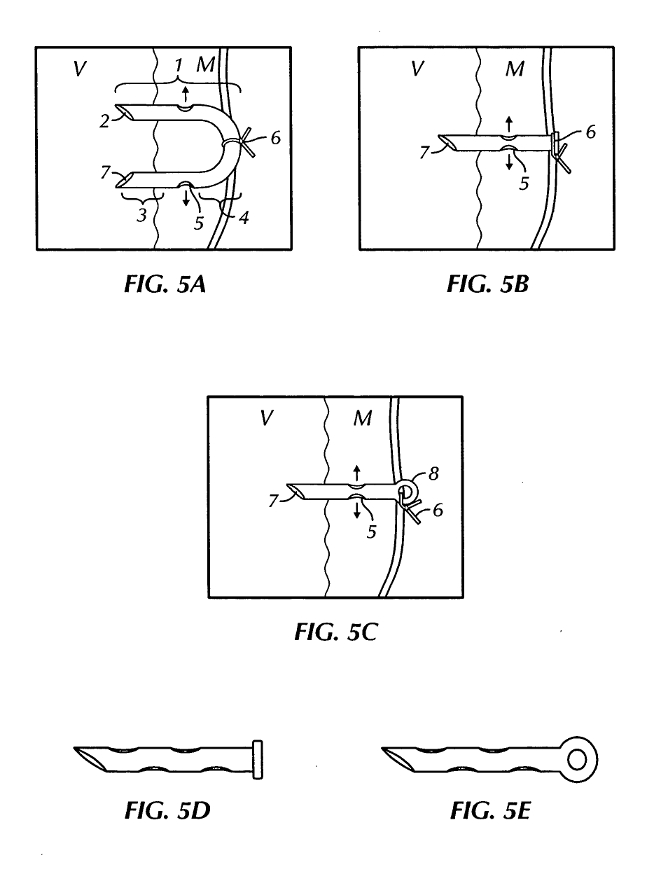

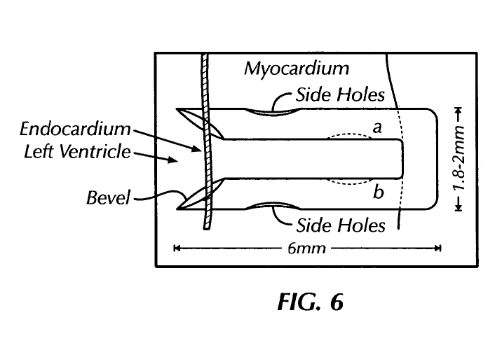

The disclosed device is adapted to provide an artificial channel that allows blood to flow directly from the left ventricle into the myocardium. As such, it is generally elongated and hollow, and may have one or more shafts. One preferred device design is depicted in FIGS. 2 and 5. As shown in FIG. 5(a), this device comprises two elongated shafts (1) which are joined together to form a horseshoe. The shaft has an aperture (2) running through it to permit blood flow through the device. The shaft has a proximal end (3) and a distal end (4). As shown, the distal ends of the shafts in the HS needle form the “bended” portion of the horseshoe. Disposed in the shaft between the proximal and distal ends is at least one opening (5) which permits blood to flow out of the device and into the myocardium as depicted by the arrows. Also shown is a suture (6) which is an exemplary means of securing the device in place to the myocardium. The proximal end (3) terminates in a hollow point (7) (i.e., a “bevel”), much like a conventional syringe needle, through which blood from the left ventricle flows into the aperture.

As shown in FIG. 5(b), an alternative device design comprises a single shaft bearing two openings (5). The “nail head” shape of this device allows it to be secured to the myocardium with a suture (6) at its distal end. In yet another embodiment shown in FIG. 5(c), the device has an “eyelet” at the distal end for securing it to the myocardium with a suture (6). As shown in FIGS. 5(d) and 5(e), these devices may have a plurality of openings along the shaft.

Preferred embodiments of the aforementioned devices are closed at the distal end, such that blood from the left ventricle is not exposed to the atmosphere during or after placement of the device, which may result in clot formation. As such, all of the openings in the device through which blood passes are contained within the ventricle or myocardium. This is an improvement over the SP device.

The disclosed devices are preferably made of any surgical material suitable to transfer oxygenated blood, such as high density synthetic materials or stainless steel. Preferred materials are those that permit the introduction of holes within the wall of the shaft, which permit the passage of oxygenated blood from the lumen or aperture of the shaft. The dimensions of the devices are easily ascertained from knowledge of the size of the heart of the recipient, and can be manufactured in a variety of different sizes that the surgeon can choose from prior to implantation.

The disclosed devices are easily adapted for use as indwelling apparatuses. The devices are preferably rigid enough at the needle points in order to penetrate the entire wall of the heart, but also flexible enough so that normal cardiac rhythm is not disrupted.

Various preferred embodiments of the disclosed devices are coated with a variety of substances. For example, substances that provide diagnostic or therapeutic features to the disclosed devices can be used to coat the devices. For example, a device can be coated with a dye that is released as blood flows through the device's shaft. In another example, a therapeutic substance is coated onto a device, where the substance is slowly released over time. Alternatively, the substances used to coat the disclosed devices can be bound to the device in such a way that holds the substance proximate to the shaft of the device.

Preferred substances that can be coated onto or within the aperture of a perfusion device include antiplatelet compounds and antithrombins. Examples of antiplatelets include aspirin (acetylsalicylic acid); choline magnesium trisalicylate; choline salicylate; clopidogrel; magnesium salicylate; persantine; salicylic acid; salsalate; sodium salicylate; and ticlopidine hydrochloride. Antithrombins are any compounds that inhibit thrombosis. Examples of antithrombins include a family of proteins belonging to the serpin superfamily that neutralizes the action of thrombin. Six naturally occurring antithrombins have been identified and are designated by Roman numerals I to VI. Of these, Antithrombin I and antithrombin III appear to be of major importance.

Other substances that can be used to coat a perfusion device include antibodies such as antibodies which bind endothelial cells, and inducers of angiogenesis such as estrogen or vascular endothelial growth factor.

The disclosed devices may take a variety of different shapes. Exemplified preferred embodiments include devices comprising straight or bent shafts. Additional preferred embodiments include devices with a plurality of shafts (e.g., 3, 4, 5, 6, or more shafts) joined such that the arms of each shaft array themselves out from a point at which the shafts converge. For example, a triple-shafted device is contemplated wherein each shaft is arrayed at one or more acute or obtuse angles from the other shafts.

The disclosed devices can be inserted into the myocardium of a subject using a variety of routes. Preferably the disclosed devices are designed to be inserted into the heart by way of a laparoscopic space, which minimizes the invasiveness of surgical placement. The disclosed devices may also be inserted into a targeted myocardium once the chest cavity has been opened, such as during open-heart surgery.

### EXAMPLES

Two needles have been designed in order to revascularize an ischemic left ventricle in the event of left coronary artery occlusion. This study was conducted by performing the Lee modified Fox-Montorsi Heart-Lung Transplant on 25 San Diego Microsurgical Institute bred Sprague Dawley rats that have been subjected to left coronary artery ligation in each case. Among these rats, a Straight-Porous (SP) needling procedure was applied to 9 heterotopically transplanted rat hearts and a distinctive Horse-Shoe (HS) shaped needle application was preformed on 16 heterotopically transplanted rat hearts. This experiment represents an acute study on the efficiency of these two needles to transmit oxygen rich blood from the left ventricle into the ischemic myocardium.

### Example 1

### Preparation of Heart-Lung Transplant Animal Model

A heart-lung transplant model was prepared as previously reported in the literature (Fox, U., and Montorsi, M. A., J. Microsurg. (1980) 1:377-380; and Lee, S., et al., Transpl. (1982) 33:438-442). The anesthetized rat was surgically prepared and the chest cavity was entered through an incision below the sternum. The superior vena cava (SVC) and azygos veins were then ligated with a 6-0 suture and cut. The inferior vena cava (IVC) was clamped and the aorta was cut at the innominate artery. Next, the heart and lungs were perfused with 0.3 to 0.5 ml of normothermic saline via the IVC. The IVC was immediately ligated and cut following perfusion to minimize the chance of blood clots. The trachea was then cut, and the entire graft was peeled away from the esophagus. Once the graft was removed from the donor, the aorta was irrigated with approximately 1 ml of saline to remove any clots and excess blood remaining in the vascular tree. The harvested lung lobes were removed by ligation of each hilum, with the exception of one lobe which served as a blood reservoir. For long-term graphs, a tracheo-bronchial resection was preformed in order to decrease the probability of a future lung infection. Aorta-aortic end-to-side anastomosis was performed in the abdominal cavity of the recipient animal using microvascular techniques.

### EXAMPLE 2

### Ischemic Heart Model

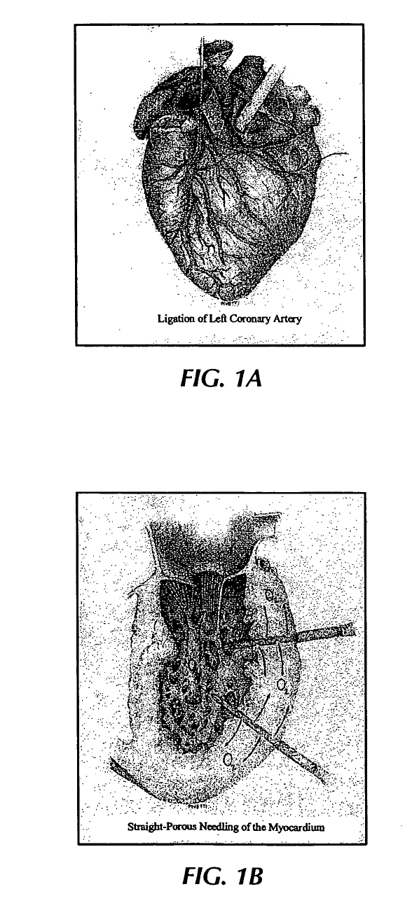

Following removal of the heart-lung graft from the donor, the aorta was cannulated with a 26-gauge blunt needle to locate the position of the left coronary artery (LCA). Once the position was found, the LCA was ligated 2-3 mm distal from the LCA take-off with a 9-0 suture which was left loose until control data was accumulated. No further studies were conducted until complete ischemia of the myocardium had taken place, which was indicated by a white discoloration of the left ventricle. This procedure is depicted in FIG. 1 (a).

Once the heart became ischemic, a 23-gauge Straight Porous (SP) needle was inserted randomly 4 to 5 times into the ischemic left ventricle from the outside as shown in FIG. 1(b). The SP needle harbored four side holes that were drilled into the shaft within a distance of 5 mm from the point of the needle. A small silicone doughnut rung was placed on the needle 6 mm away from the point in order to ensure that only the thickness of the myocardium was pierced. The needle was inserted into the pale, avascular area of the myocardium and was held in place for thirty seconds. During this time, contraction of the heart forced oxygenated ventricular blood into the needle and then out through the side holes into the ischemic myocardium.

A parallel study was performed as described above using the Horse Shoe (HS) needle as depicted in FIG. 2. The HS needle shown in FIG. 2(a) has two large oval shaped side holes in the needle's shaft. FIG. 2(b) depicts a mammalian heart with ligation of the LCA and the position of the HS Needle in the myocardium. FIG. 2(c) depicts a magnified cross-section of the left ventricle that demonstrates the function of the HS needle. As shown, oxygenated blood enters the needle through both needle points and then passes out of the two side holes into the myocardium.

The HS needle was prepared as follows: a 23-gauge needle was bent in a right angle 5.5 mm from the needle point and was bent a second time in another right angle at approximately 2 mm from the first bend, so as to make two parallel shafts. Oval shaped side holes were drilled in both shafts of the HS Needle 1 mm distal to each right angle bend in the needle. The remaining needle shaft was cut at a distance of 5.5 mm from the second bend and the cut end was ground down to make a needlepoint (FIG. 2a). This needle was inserted into the ischemic myocardium 3 mm distal to the ligation site of the LCA, and held in place with a 9-0 suture (FIG. 2b). Contraction of the heart forced oxygenated blood into both shafts of the needle and out the two large side holes into the myocardium (FIG. 2c). Furthermore, the HS needle provided a closed environment for blood to circulate in the myocardium, thus greatly minimizing the probability of air exposure and blood clots.

Doppler readings were taken by placing a 2 mm probe on the LCA once a normal heart beat was established. The Doppler machine (Koven Technology, Inc., St. Louis, Mo.) was used to record the peak (PK) and mean (MN) kHz values of LCA blood flow as well as heart rate. Doppler readings were taken at each stage of the procedure, which consisted of pre-ligation of LCA (control), ligation of LCA, and needling of the left ventricle. All data from these readings were averaged by sex.

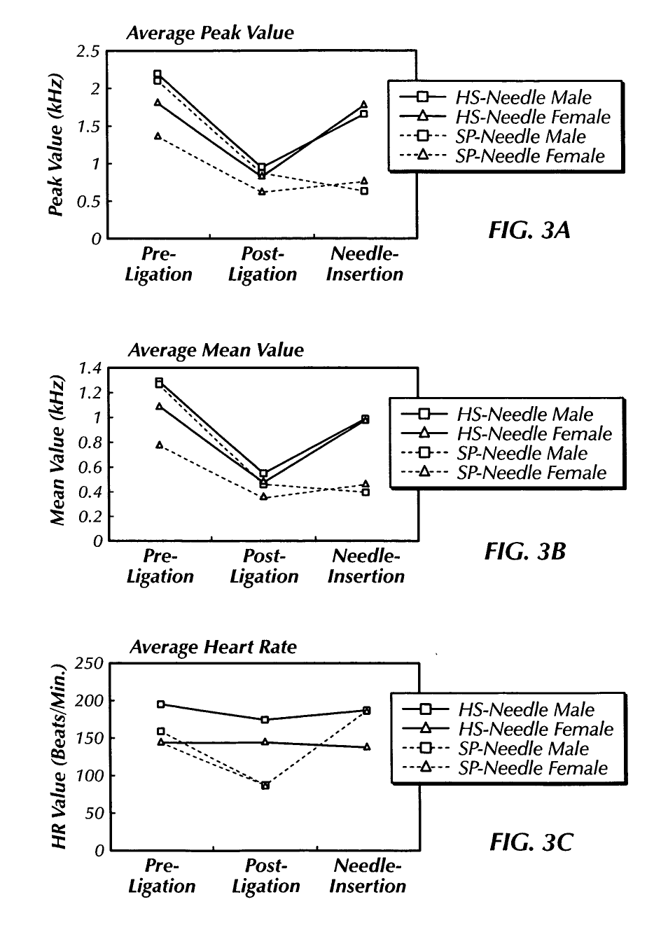

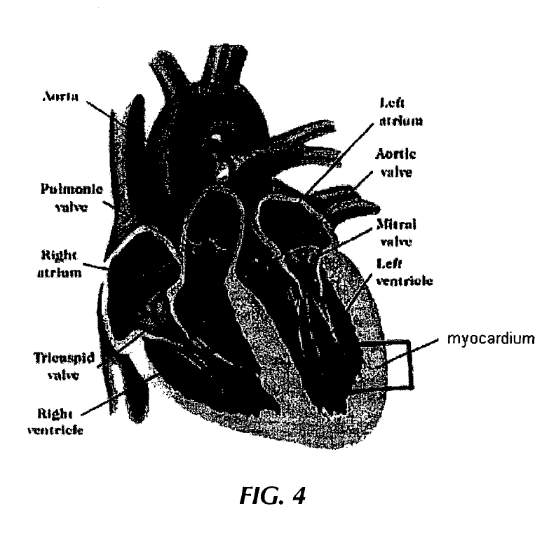

A graphical analysis of the Doppler results using the SP and HS needles is shown in FIG. 3. The stages of the procedure are shown on the x-axis and the graphs are divide up into three groups: (a) average peak value, (b) average mean value, and ( c) average heart rate. As shown, superior results were achieved using the HS needle.

Comparison of the male vs. female pre-ligation (control) data indicates that the PK and MN kHz values was on the average of 0.20 kHz higher in males than in females (FIGS. 3a and 3b). However, the male vs. female pre-ligation data for heart rate shows that the two sexes have a similar heart rate at approximately 150 beats per minute (FIG. 3c).

Ligation of the LCA produced a dramatic white discoloration of the left myocardium that had an area of approximately 5 mm in diameter. Doppler readings taken after ligation of the LCA confirmed that the heart had become ischemic due to a dramatic decrease in PK and MN kHz values. FIGS. 3a and 3b clearly show that the PK and MN kHz ligation data for both sexes from the SP Needle group and from the HS Needle group dropped on the average of 60% from the control data. The most significant case being the male SP Needle group whose MN kHz value decreased from 1.25 to 0.38 kHz, which was a 70% drop from the original value (Table 1b, below). Furthermore, ligation of the LCA appeared to have little effect on heart rate values.

Results of the SP Needle procedure show that the males were only able to recover 30% of the pre-ligation peak kHz value and 26% of the pre-ligation mean kHz value. On the other hand, the females in this group were able to recover approximately 50% of the PK and MN control values (FIGS. 3a and 3b). Visual observations concluded that immediately following the SP needling procedure the white discoloration of the left myocardium disappeared. However, after 5 minutes post operation time the needled area of the myocardium became black in color. This observation indicated that blood clots had developed in the myocardium, most likely due to air exposure from the open puncture wounds created by the SP Needle. Furthermore, the presence of clots in the myocardium would account for the minimal recovery seen in the PK and MN kHz values recorded after completion of the SP needling procedure.

It is also important to point out that the SP needling procedure had a dramatic affect on heart rate. Heart rate values for both sexes in the SP Needle group increased to levels higher than the original control values (FIG. 3c). This data indicates that the insertion and removal of the SP Needle into the left ventricle acted as a mechanical stimulator of the myocardial muscular bundles that increased heart rate.

Upon insertion of the HS Needle into an ischemic myocardium the Doppler showed results that the males were able to recover 75% of the control PK kHz value and 73% of the control MN kHz value. The female HS Needle group had an even larger recovery of the control values at 97% PK kHz and 89% MN kHz (Tables 1a and 1b, below). Furthermore, application of the HS needle caused the white, discolored area of the ischemic myocardium to disappear. However, unlike the SP Needle, the HS Needle did not produce any blood clots in the myocardium at 5 min. post operation time. This observation indicated that the HS Needle did indeed provide a closed environment for blood to circulate in the myocardium, and that air exposure into the puncture wounds was greatly minimized. HS needling had little effect on heart rate as well; whereas, heart rate values stayed at 96% relative to the original control values. This data can be attributed to the fact that the HS needle is used to puncture the myocardium only once during the procedure.

TABLE 1
Doppler Results

| | LCA | | |
|---|---|---|---|
| | Pre-ligation | | Needle |
| Group | (Control) | Ligation | Insertion |

Average Peak Value (Mean kHZ ± S.D.)

| | | | | |
|---|---|---|---|---|
| a. | HS-Needle Male | 2.20 ± 0.25 | 0.92 ± 0.28 | 1.64 ± 0.46 |
| | HS-Needle | 1.81 ± 0.52 | 0.80 ± 0.22 | 1.76 ± 0.49 |
| | Female | | | |
| | SP-Needle Male | 2.11 ± 0.17 | 0.83 ± 0.15 | 0.61 ± 0.05 |
| | SP-Needle | 1.35 ± 0.14 | 0.62 ± 0.16 | 0.72 ± 0.11 |
| | Female | | | |

Average Mean Value (Mean kHZ ± S.D.)

| | | | | |
|---|---|---|---|---|
| b. | HS-Needle Male | 1.29 ± 0.28 | 0.49 ± 0.14 | 0.95 ± 0.35 |
| | HS-Needle | 1.06 ± 0.40 | 0.42 ± 0.09 | 0.95 ± 0.36 |
| | Female | | | |
| | SP-Needle Male | 1.25 ± 0.22 | 0.38 ± 0.11 | 0.33 ± 0.05 |
| | SP-Needle | 0.72 ± 0.08 | 0.29 ± 0.08 | 0.40 ± 0.09 |
| | Female | | | |

Average Heart Rate (Mean Beats/Min. ± S.D.)

| | | | | |
|---|---|---|---|---|
| c. | HS-Needle Male | 191.7 ± 30.3 | 173.0 ± 38.4 | 184.8 ± 25.8 |
| | HS-Needle | 143.0 ± 40.1 | 145.0 ± 59.0 | 137.3 ± 18.4 |
| | Female | | | |
| | SP-Needle Male | 143.0 ± 9.8 | 89.7 ± 10.1 | 186.3 ± 8.5 |
| | SP-Needle | 158.4 ± 35.7 | 85.4 ± 14.1 | 185.6 ± 26.0 |
| | Female | | | |

### EXAMPLE 3

### Laparoscopic Insertion of a Device for Externally Effecting Perfusion of Oxygenated Blood into the Ischemic Myocardium

A subject presenting symptoms of an ischemic myocardium is prepared for the laparoscopic insertion of a perfusion device. A small video camera and laparoscopic instruments are inserted into the chest of the subject through a small incision. Once the incision is made, a needle is inserted into the subject to begin insufflation. Once satisfactory insufflation is achieved, a trocar is inserted through the incision.

Once the insertion site is established, a delivery device comprising the perfusion device is laparoscopically introduced through the fibrous pericardium and the pericardial cavity, and into the myocardium. After insertion of the perfusion device into the myocardium, it is sutured into place. Contraction of the heart forces oxygenated blood into the shaft of the perfusion device and out through the holes in the shaft and thus into the myocardium. Insertion of the perfusion device quickly alleviates the ischemia in the general region in which the device is inserted.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. An implantable device for external effecting perfusion of oxygenated blood into myocardial tissue from a left ventricle, comprising:
a shaft having an aperture running longitudinally therethrough, the shaft further comprising a proximal end terminating in a point and a distal end adapted to be detachably secured to the outside of the myocardial tissue, wherein the proximal end and the distal end are spaced to allow the proximal end to enter the left ventricle, and wherein the shaft further comprises at least one opening into the aperture between the proximal end and the distal end.

2. The device of claim 1, wherein the shaft is curved.

3. The device of claim 2, wherein the shaft is U-shaped.

4. The device of claim 1, wherein the shaft is straight.

5. The device of claim 4, wherein the shaft is blunted on the distal end.

6. The device of claim 1, wherein the aperture is coated with a substance.

7. The device of claim 6, wherein the substance is selected from the group consisting of an antibody, an inducer of angiogenesis, an antiplatelet agent and an antithrombin agent.

8. The device of claim 7, wherein the inducer of angiogenesis is human vascular endothelial growth factor.

9. The device of claim 7, wherein the antibody binds to endothelial cells.

10. The device of claim 6, wherein the substance elutes from the aperture.

11. A method of implanting a perfusion device for externally effecting perfusion of oxygenated blood into myocardial tissue of a heart's left ventricle, comprising:
providing the perfusion device, wherein the perfusion device comprises a shaft, an aperture running longitudinally through the shaft, wherein the shaft further comprises a proximal end terminating in a point and a distal end adapted to be detachably secured to the outside of the myocardial tissue, wherein the shaft further comprises at least one opening into the aperture between the proximal end and the distal end; and
inserting the device into myocardial tissue, such that oxygenated blood is perfused into the myocardial tissue from the left ventricle.

12. The method of claim 11, wherein the shaft is curved.

13. The method of claim 11, wherein the shaft is straight.

14. The method of claim 11, wherein the aperture is coated with a substance selected from the group consisting of an antibody, an inducer of angiogenesis, an antiplatelet agent and an antithrombin agent.

15. A method of treating an ischemic myocardium, comprising:
identifying a subject suffering from an ischemic myocardium;
inserting a perfusion device for externally effecting perfusion of oxygenated blood into the ischemic myocardium, whereby oxygenated blood is perfused into the ischemic myocardium from the left ventricle, wherein the perfusion device comprises a shaft having an aperture running longitudinally therethrough, the shaft further comprising a proximal end terminating in a point and a distal end adapted to be detachably secured to the outside of the myocardial tissue, wherein the proximal end and the distal end are spaced to allow the proximal end to enter the left ventricle, and wherein the shaft further comprises at least one opening into the aperture between the proximal end and the distal end.

16. The method of claim 15, wherein the device is inserted into the ischemic myocardium using an insertion tool.

17. The method of claim 15, wherein the device is inserted laparoscopically.

18. The method of claim 15, wherein the shaft is curved.

19. The method of claim 15, wherein the shaft is straight.

20. The method of claim 15, wherein the aperture is coated with a substance selected from the group consisting of an antibody, an inducer of angiogenesis, an antiplatelet agent and an antithrombin agent.
